(19) United States
(12) Patent Application Publication
(10) Pub. No.: US 2013/0123886 A1
(43) Pub. Date: May 16, 2013

(54) WEARABLE HORSE COOLING DEVICE

(71) Applicants: Gary CHIU, Plano, TX (US); Naijian Fu, Fuzhou (CN)

(72) Inventors: Gary CHIU, Plano, TX (US); Naijian Fu, Fuzhou (CN)

(21) Appl. No.: 13/296,405

(22) Filed: Nov. 15, 2011

(51) Int. Cl. A61F 7/10; A61D 9/00

(52) U.S. Cl. 607/104

(57) ABSTRACT

A wearable horse cooling device has at least one cold water circulating module and a fixing module. Each one of the at least one cold water circulating module has a cooling unit, a wrap and a connection hose assembly. The wrap has a water bag and an air bag for generating a holding force. The connection hose assembly is connected between and communicates with each cooing unit and the water bag of the wrap. The fixing module is securely connected with each one of the at least one cold water circulating module and tied to the body of a horse to bind the cold water circulating unit on the body of the horse so that the cold water circulating unit can be firmly mounted and worn on the body of the horse without causing discomfort of the horse.

### BACKGROUND OF THE INVENTION

1. Field of the Invention

The present invention relates to a horse cooling device, and more particularly to a wearable horse cooling device to lower the temperature of and apply cold compresses to a horse.

2. Description of the Related Art

Horse cooling devices are more commonplace in western world and are available to lower the temperature of and applying cold compresses to horses, especially useful in applying cold compresses as horses usually exercise strenuously and easily get hurt.

The principle of a horse cooling device is that low temperature cold water in a container is pumped into a water inlet of a water bag by a water pump and is circulated back to the container through a water outlet of the water bag. Thus, outside surfaces of the water bag can be kept at low temperature. The water bag is attached to a portion of the body of a horse requiring a temperature drop or a cold compress and is strapped and fastened on the body of the horse to constantly lower temperature of or apply cold compresses to the horse.

Conventional horse cooling devices tend to bulky and heavy, need to be placed beside horses, and are connected to the cooling water bags fastened on the bodies of horses through water pipes and air pipes. When such a horse cooling device is applied to cool temperature of or apply a cold compress to a horse, the horse must be confined in a tiny space to prevent the horse from running around. Otherwise, wild horse movement can break off the water pipes and air pipes connected between a cooling unit and the water bag and even pull and damage the cooling unit and the water bag. Moreover, due to prolonged confinement in a tiny space and limited movement, horses easily get irritable and fiercely kick and jump at the cost of horses' health and smoothness of cold compress process.

### SUMMARY OF THE INVENTION

An objective of the present invention is to provide a wearable horse cooling device to lower the temperature of and apply cold compresses to a horse.

To achieve the foregoing objective, the wearable horse cooling device has at least one cold water circulating module and a fixing module.

Each one of the at least one cold water circulating module has a cooling unit, a wrap, a connection hose assembly and a charging unit.

The wrap has a water bag and an air bag mounted therein. The air bag is inflated by the cooling unit to be adapted to hold a portion of a horse.

The connection hose assembly is connected between and communicates with the cooling unit and the water bag and the air bag of the wrap so that the cooling unit circulates cold water between the water bag of the wrap and the cooling unit and inflates or deflates the air bag.

The charging unit has a battery set and is connected to an AC mains to charge the battery set adapted to supply an operating power to the wearable horse cooling device.

The fixing module is securely connected with each one of the at least one cold water circulating module and is adapted to be tied to a body of a horse for each one of the at least one cold water circulating unit to be mounted on the body of the horse.

Preferably, the fixing module has an adjustable neck strap and two adjustable girth straps.

Preferably, the wearable horse cooling device has two cold water circulating modules, the two cooling units of the two cold water circulating modules are connected with each other by using a connection strap mounted between the two cooling units so that the two cooling units is adapted to be mounted on two sides of a horse with the connection strap striding across a back of the horse.

Preferably, the connection strap is made of a soft and resilient material.

Preferably, each cooling unit has two buckles mounted on a bottom of the cooling unit, each buckle on one of the cooling units engages one of the buckles on the other cooling unit so that the bottoms of the cooling units is adjoined to each other.

Preferably, each cooling unit has a handle mounted on a top of the cooling unit, the wearable horse cooling device further has a Velcro fastener mounted on the handle of one of the cooling units so that the two handles of the cooling units are jointly wrapped around and bundled together by the Velcro fastener.

Preferably, the wearable horse cooling device further has a carry-on bag for the at least one cold water circulating module and the fixing module to be accommodated in the carry-on bag.

Preferably, the connection hose assembly has an input water hose, an output water hose and an air hose, and the cooling unit has an ice container, a water pump, a pneumatic pump and an electronic controller. The ice container is connected to the water bag of the wrap through the input water hose. The water pump is connected to the ice container and to the water bag of the wrap through the output water hose of the connection hose assembly. The pneumatic pump is connected to the air bag of the wrap through the air hose of the connection hose assembly. The electronic controller controls the water pump to circulate cold water between the ice container and the water bag of the wrap through the input water hose and the output water hose and controls the pneumatic pump to inflate or deflate the air bag through the air hose.

The wearable horse cooling device has the advantages of being firmly mounted on the back of a horse without causing discomfort of the horse, and allowing the horse carrying the wearable horse cooling device to freely move. Accordingly, the wearable horse cooling device solves the inconvenience of conventional horse cooling device during operation and can be accommodated in a carry-on bag to facilitate the carrying need and storage.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a wearable horse cooling device in accordance with the present invention mounted on a house;

FIG. 2 is a perspective view of two cooling units of the wearable horse cooling device in FIG. 1;

FIG. 3 is a front view of an unfolded wrap of the wearable horse cooling device in FIG. 1;

FIG. 4 is an operational perspective view of the wearable horse cooling device in FIG. 1 mounted on a horse;

FIG. 5 is another operational perspective view of the wearable horse cooling device in FIG. 1 mounted on a horse;

FIG. 6 is a perspective view of a cold water circulating module of the wearable horse cooling device in FIG. 1;

FIG. 7 is a first operational perspective view of the cold water circulating module t in FIG. 6;

FIG. 8 is a second operational perspective view of the cold water circulating module in FIG. 6; and

FIG. 9 is a third operational perspective view of the cold water circulating module in FIG. 6.

### DETAILED DESCRIPTION OF THE INVENTION

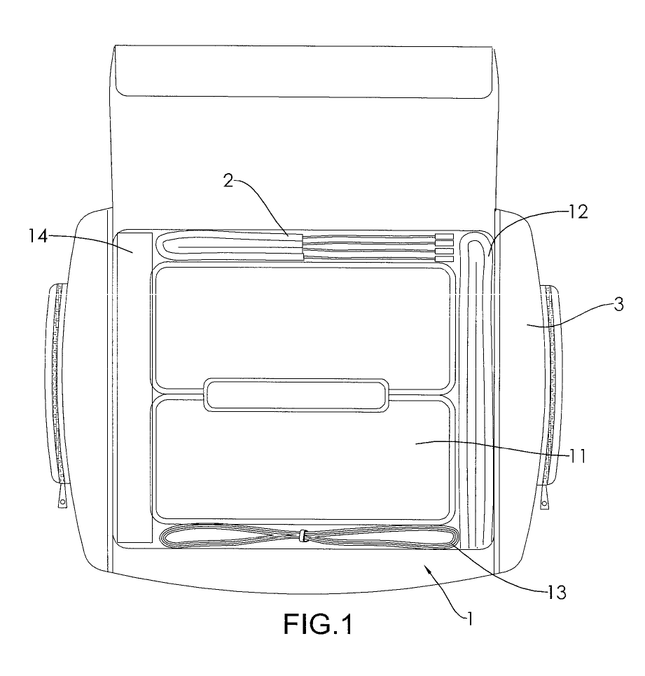

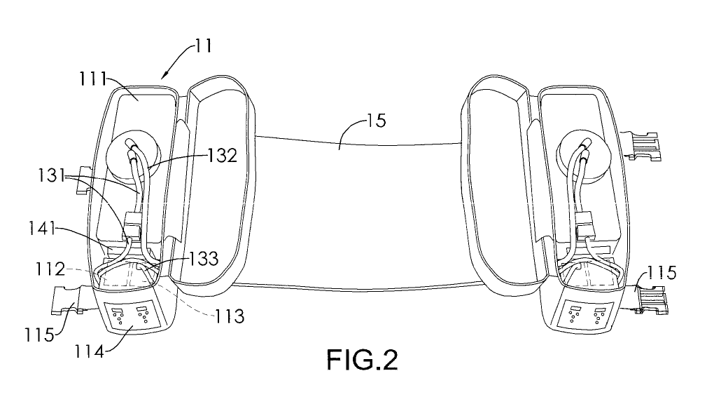

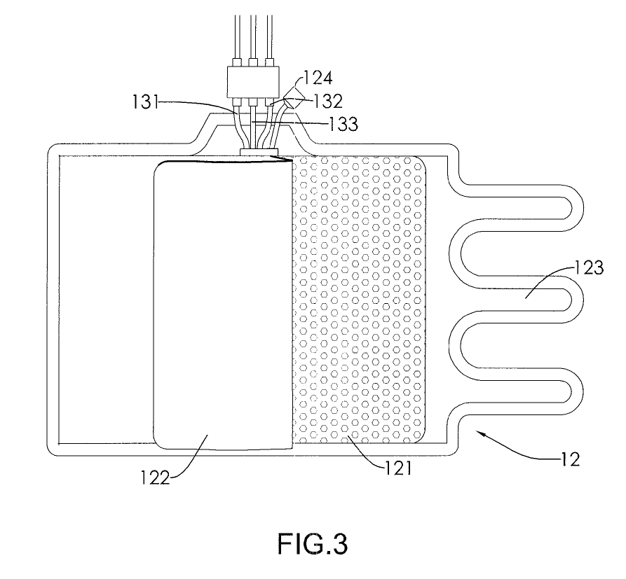

With reference to FIGS. 1 to 5, a wearable horse cooling device in accordance with the present invention has at least one cold water circulating module 1 and a fixing module 2. In the present embodiment, the wearable horse cooling device has two cold water circulating modules 1. Each cold water circulating module 1 has a cooling unit 11, a wrap 12, a connection hose assembly 13 and a charging unit 14. The cooling unit 11 has an ice container 111, a water pump 112, a pneumatic pump 113, and an electronic controller 114. The wrap 12 has a water bag 121, an air bag 122 and a fastener 123. The water bag 121 and the air bag 122 are mounted in the wrap 12. The air bag 122 has an automatic air release valve 124. The automatic air release valve 124 is connected and communicates with the air bag 122 and serves to automatically release the air overcharged in the air bag 122 to prevent a wearing portion of the horse from being over-tightened or the air bag 122 from being blown up. The fastener 123 serves to be fastened around a portion of a horse. In the present embodiment, the wrap 12 is a leg wrap, and the fastener 123 is a Velcro fastener. The connection hose assembly 13 has an input water hose 131, an output water hose 132 and an air hose 133. The input water hose 131 and the output water hose 132 are connected between and communicates with the water pump 112 of the cooing unit 11 and the water bag 121 of the wrap 12 of one of the cold water circulating modules 1. The air hose 133 is connected between and communicates with the pneumatic pump 113 of the cooling unit 11 of each cold water circulating module 1 the and the air bag 122 of the wrap 12. The electronic controller 114 controls the water pump 112 to circulate cold water between the ice container 111 and the water bag 121 through the input water hose 131 and the output water hose 132 and controls the pneumatic pump 113 to inflate or deflate the air bag 122 through the air hose 133. When inflated or deflated, the air bag 122 holds or loosens a portion of a horse. The charging unit 14 is connected to the AC mains to charge a battery set 141 thereof for supplying an operating power to the wearable horse cooling device. The fixing module 2 is securely connected with the cold water circulating modules 1 and is tied to the body of a horse so as to firmly mount the cold water circulating unit 1 on the body of the horse without causing discomfort of the horse. In the present embodiment, the fixing module 2 has an adjustable neck strap 21 and two adjustable girth straps 22.

In the present embodiment, the two cooling units 11 of the two cold water circulating modules 1 are connected with each other by using a connection strap 15 mounted between the two cooling units 11 so that the two cooling units 11 can be mounted on two sides of a horse with the connection strap 15 striding across the horse back. The connection strap 15 is made of a soft and resilient material to ensure the horse's wearing comfort.

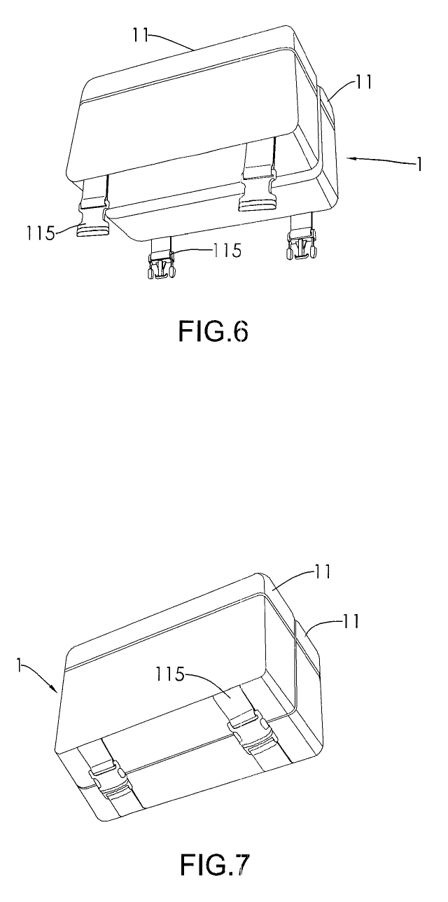

With reference to FIGS. 6 and 7, each cooling unit 11 has two buckles 115 mounted on a bottom of the cooling unit 1. Each buckle 115 on one of the cooling units 11 may engage one of the buckles 115 on the other cooling unit 11 so that the bottoms of the cooling units 1 can be adjoined to each other to make the cooling units 1 more portable or may engage one end of one of the adjustable girth strap 22 of the fixing module 2 to facilitate fixing the cooling units 11.

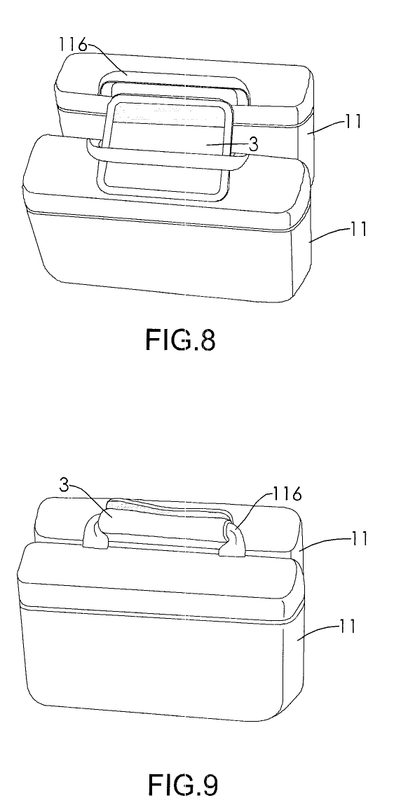

With reference to FIGS. 8 and 9, each cooling unit 11 has a handle 116 mounted on a top of the cooling unit 11. The wearable horse cooling device further has a Velcro fastener 3 mounted on the handle 116 of one of the cooling units 11 so that the two handles 116 of the cooling units 11 can be jointly wrapped around and bundled together by the Velcro fastener 3 to facilitate the cooling units 11 to be easily carried.

With further reference to FIG. 1, for the convenience of carrying around and storage, the wearable horse cooling device further has a carry-on bag 3 for the two cold water circulating modules 1 and the fixing module 2 to be accommodated in the carry-on bag 3.

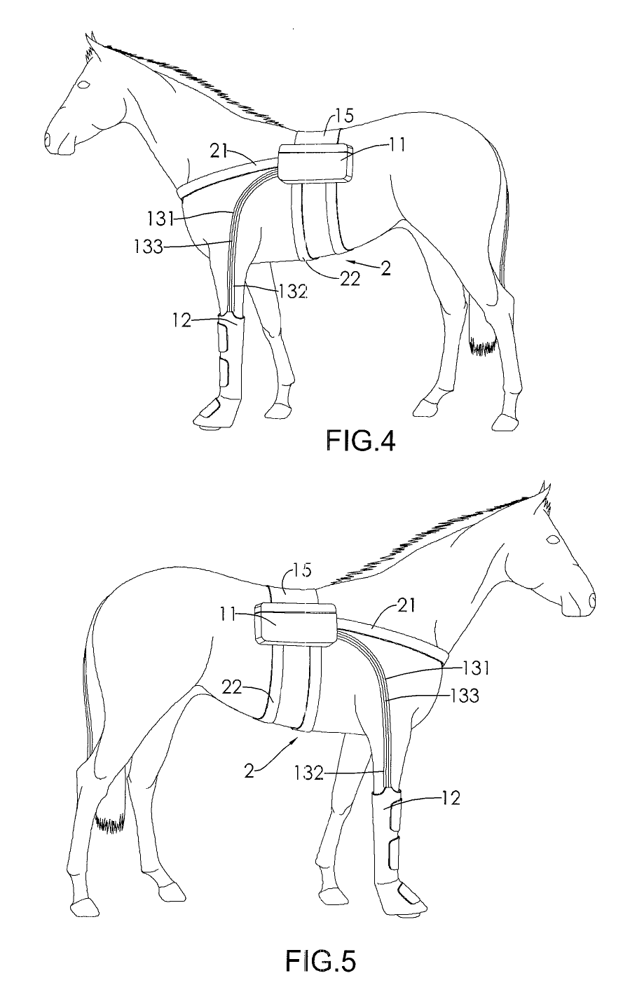

With further reference to FIGS. 4 and 5, before the wearable horse cooling device is operated, the charged battery set 142 is electrically connecting with the cooling unit 11 of each cold water circulating module 1, the cooling unit 11 of each cold water circulating module 1 of the wearable horse cooling device is placed on a body of a horse, the two adjustable girth straps 22 are securely connected to the cooling unit 11 of each cold water circulating module 1 and are tightened to fit under the horse so that the cooling unit 11 of each cold water circulating module 1 can be firmly mounted on the body of the horse, the adjustable neck strap 21 is securely connected to the cooling unit 11 of each cold water circulating module 1 and is tightened, the input water hose 131 of the connection hose assembly 13 is connected between the water bag 121 of the wrap 12 and the ice container 111 of the cooling unit 11 of each cold water circulating module 1, the output water hose 132 is connected with the water pump 112 and the water bag 121 of the wrap 12, the air hose 133 of the connection hose assembly 13 is connected between the air bag 122 of the wrap 12 and the pneumatic pump 113 of the cooling unit 11 of each cold water circulating module 1, the ice container 111 of the cooling unit 11 of each cold water circulating module 1 is filled with ice cubes and clean water, the wrap 12 is loosely mounted around a portion of the horse and the pneumatic pump 113 is activated to inflate the air bag 122 of the wrap 12 to properly hold the portion of the horse, and the water pump 112 is continuously turned on and off for a while until the water bag 121 of the wrap 12 of each cold water circulating module 1 is fully filled with cold water circulated from the ice container 111 of the cold water circulating module 1. After the foregoing preparation processes are completed, operation parameters required for a cooling cycle, such as a pressure value of the air bag 122 for the wrap 12 of each cold water circulating module 1 to hold a portion of a horse and a time period of a cooling cycle, are selected according to a cooling need and the cooling cycle is operated according to the operation parameters.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A wearable horse cooling device comprising:
at least one cold water circulating module, each one of the at least one cold water circulating module having:
a cooling unit;
a wrap having a water bag and an air bag mounted therein, wherein the air bag is inflated by the cooling unit to be adapted to hold a portion of a horse;
a connection hose assembly connected between and communicating with the cooling unit and the water bag and the air bag of the wrap so that the cooling unit circulates cold water between the water bag of the wrap and the cooling unit and inflates or deflates the air bag; and
a charging unit having a battery set and connected to an AC mains to charge the battery set adapted to supply an operating power to the wearable horse cooling device; and
a fixing module securely connected with each one of the at least one cold water circulating module and adapted to be tied to a body of the horse for each one of the at least one cold water circulating unit to be mounted on the body of the horse.

2. The wearable horse cooling device as claimed in claim 1, wherein the fixing module has an adjustable neck strap and two adjustable girth straps.

3. The wearable horse cooling device as claimed in claim 1, wherein the wearable horse cooling device has two cold water circulating modules, the two cooling units of the two cold water circulating modules are connected with each other by using a connection strap mounted between the two cooling units so that the two cooling units is adapted to be mounted on two sides of a horse with the connection strap striding across a back of the horse.

4. The wearable horse cooling device as claimed in claim 3, wherein the connection strap is made of a soft and resilient material.

5. The wearable horse cooling device as claimed in claim 3, wherein each cooling unit has two buckles mounted on a bottom of the cooling unit, each buckle on one of the cooling units engages one of the buckles on the other cooling unit so that the bottoms of the cooling units is adjoined to each other.

6. The wearable horse cooling device as claimed in claim 3, wherein each cooling unit has a handle mounted on a top of the cooling unit, the wearable horse cooling device further has a Velcro fastener mounted on the handle of one of the cooling units so that the two handles of the cooling units are jointly wrapped around and bundled together by the Velcro fastener.

7. The wearable horse cooling device as claimed in claim 1, further comprising a carry-on bag for the at least one cold water circulating module and the fixing module to be accommodated in the carry-on bag.

8. The wearable horse cooling device as claimed in claim 1, wherein the connection hose assembly has:
an input water hose;
an output water hose; and
an air hose;
the cooling unit has:
an ice container connected to the water bag of the wrap through the input water hose;
a water pump connected to the ice container and to the water bag of the wrap through the output water hose of the connection hose assembly;
a pneumatic pump connected to the air bag of the wrap through the air hose of the connection hose assembly; and
an electronic controller controlling the water pump to circulate cold water between the ice container and the water bag of the wrap through the input water hose and the output water hose and controlling the pneumatic pump to inflate or deflate the air bag through the air hose.

9. The wearable horse cooling device as claimed in claim 1, wherein the air bag has an automatic air release valve connected and communicates with the air bag to automatically release the air overcharged in the air bag.
